(19) United States
(12) Patent Application Publication
(10) Pub. No.: US 2023/0118020 A1
(43) Pub. Date: Apr. 20, 2023

(54) DATA GENERATION APPARATUS, DATA GENERATION METHOD, AND RECORDING MEDIUM

(71) Applicant: NEC Corperation, Minato-ku,Tokyo (JP)

(72) Inventors: So YAMADA, Tokyo (JP); Junko WATANABE, Tokyo (JP); Riki ETO, Tokyo (JP); Hiromi SHIMIZU, Tokyo (JP); Noriyuki TONOUCHI, Tokyo (JP)

(73) Assignee: NEC Corperation, Minato-ku,Tokyo (JP)

(21) Appl. No.: 17/909,625

(22) PCT Filed: Mar. 17, 2020
(86) PCT No.: PCT/JP2020/011634

§ 371 Date: Sep. 6, 2022

(51) Int. Cl. G01N 15/06; G01N 33/00

(52) U.S. Cl. CPC G01N 15/0606; G01N 33/0001

(57) ABSTRACT

In a data generation apparatus, an acquisition unit acquires original data which are odor data measured in a specific environment. A generation unit performs a linear transformation with respect to the original data, and generates augmented data which are odor data in an environment where temperature and humidity are different from those in the specific environment.

### TECHNICAL FIELD

The present disclosure relates to an augmentation of odor data measured using a sensor.

### BACKGROUND ART

A technique for detecting odor using a sensor is known. As an odor sensor, for example, a semiconductor type sensor, a crystal oscillation type sensor, a membrane type surface stress sensor and the like are known. Patent Document 1 describes a technique for measuring a sample gas using a nanomechanical sensor provided with a receptor layer, and discriminating a type of a sample gas.

### PRECEDING TECHNICAL REFERENCES

### Patent Document

Patent Document 1: Japanese Laid-open Patent Publication No. 2017-156254

### SUMMARY

### Problem to be Solved by the Invention

Based on odor data detected by an odor sensor, it is possible to predict a substance that causes an odor. Concretely, a predictive model which learned features of odor data by machine learning or the like is generated, and it is possible to predict the substance from the odor data actually detected using the predictive model. Not limited to the prediction of the substance, it is also possible to predict a sugar content from a smell of a fruit, and to predict a cancer or a health condition from an odor of a urine, for example. In this case, a large amount of training data is required to train the predictive model. Especially, in order to enable a prediction in various environments, it is necessary to conduct training of the predictive model using training data obtained under the various environments. However, it is difficult to prepare a large amount of training data by actually making measurements under every environment.

It is one object of the present disclosure to generate sets of odor data corresponding to various environments by augmenting the odor data.

### Means for Solving the Problem

According to an example aspect of the present disclosure, there is provided a data generation apparatus including:

an acquisition unit configured to acquire original data which are odor data measured in a specific environment; and

a generation unit configured to perform a linear transformation with respect to the original data, and generate augmented data which are odor data in an environment where temperature and humidity are different from that in the specific environment.

According to another example aspect of the present disclosure, there is provided a data generation method, including:

acquiring original data which are odor data measured in a specific environment; and

performing a linear transformation with respect to the original data, and generating augmented data which are odor data in an environment where temperature and humidity are different from that in the specific environment.

According to still another example aspect of the present disclosure, there is provided a recording medium storing a program, the program causing a computer to perform a process comprising:

acquiring original data which are odor data measured in a specific environment; and

performing a linear transformation with respect to the original data, and generating augmented data which are odor data in an environment where temperature and humidity are different from that in the specific environment.

### Effect of the Invention

According to the present disclosure, it becomes possible to generate sets of odor data corresponding to various environments by augmenting the odor data.

### BRIEF DESCRIPTION OF THE DRAWINGS

FIG. 1 illustrates a configuration of a data augmentation system according to a first example embodiment of the present disclosure.

FIG. 2 schematically illustrates a principle of an odor measurement apparatus.

FIG. 3 is a diagram for explaining a time constant spectrum.

FIG. 4 illustrates an example of a change due to temperature in a waveform of the time constant spectrum.

FIG. 5 illustrates an example of a change due to humidity in the waveform of the time constant spectrum.

FIG. 6 illustrates a hardware configuration of the data augmentation apparatus.

FIG. 7 illustrates a functional configuration of the data augmentation apparatus.

FIG. 8 illustrates an example of an operation matrix.

FIG. 9 illustrates an example of a data augmentation using the operation matrix.

FIG. 10 schematically illustrates an example of the data augmentation.

FIG. 11 is a flowchart of a data augmentation process.

FIG. 12 is a diagram for explaining an operation matrix according to a modification.

FIG. 13 illustrates a functional configuration of the data augmentation apparatus according to the modification.

FIG. 14 illustrates a functional configuration of a data generation apparatus according to a second example embodiment.

### EXAMPLE EMBODIMENTS

In the following, example embodiments will be described with reference to the accompanying drawings.

### First Example Embodiment

[Overall Configuration]

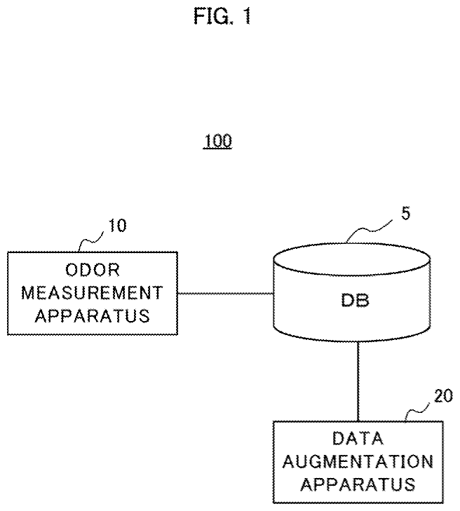

FIG. 1 illustrates a configuration of a data generation system according to a first example embodiment of the present example embodiment. The data generation system 100 includes an odor measurement apparatus 10, a database (hereinafter, also referred to as a “DB”) 5, and a data augmentation apparatus 20. The odor measurement apparatus 10 measures an odor of an object, and outputs odor data. The odor data are temporarily stored in the DB 5. The data augmentation apparatus 20 performs data augmentation using data stored in the DB 5, and stores the obtained odor data (hereinafter, also referred to as “augmented data”) in the DB 5. Specifically, the data augmentation apparatus 20 generates, as augmented data, odor data in an environment where temperature or humidity is different from that in a measurement environment of odor data measured by the odor measurement apparatus 10. By generating the augmented data using the data augmentation apparatus 20, even in a case where the measurement is not actually performed, it is possible to generate the augmented data corresponding to an environment in which the temperature or humidity is different from that concerning the measured data (hereinafter, also referred to as “original data”).

[Odor Measurement Apparatus]

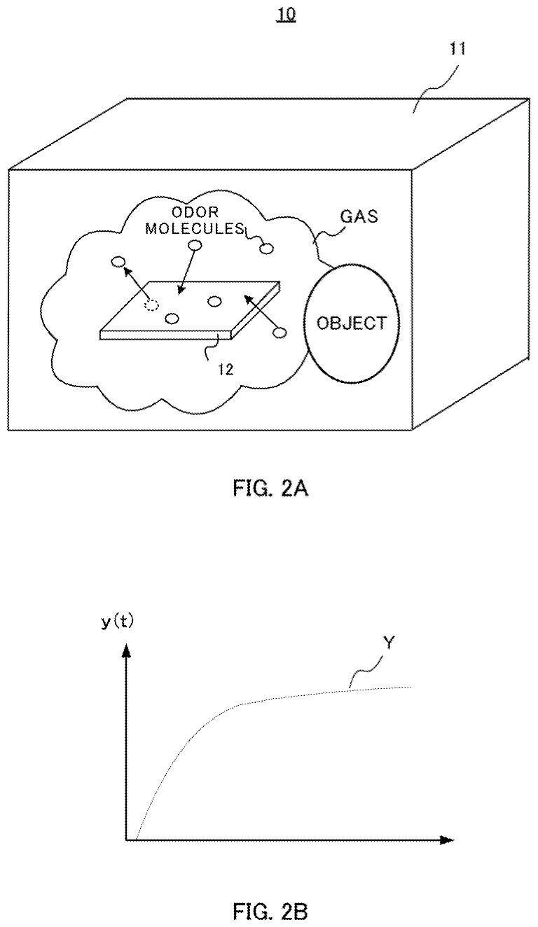

The odor measurement apparatus 10 measures an odor of an object using a sensor, and outputs odor data. FIG. 2A schematically illustrates a principle of the odor measurement apparatus 10. The odor measurement apparatus 10 includes a housing 11 and a sensor 12 disposed in the housing 11. The sensor 12 has a receptor to which an odor molecule attaches, and a detected value changes in response to an attachment and a detachment of the molecule at that receptor. The object to be a subject for an odor measurement is disposed in the housing 11. Odor molecules contained in a gas present in the housing 11 attach to the sensor 12. Hereinafter, the gas being sensed by the sensor 12 is referred to as a “target gas”. Furthermore, time series data of the detected value, which is output from the sensor 12, are represented by “time series data Y”. When the detected value of a time t of the time series data Y is denoted as y(t), as illustrated in FIG. 2B, the time series data Y are a vector formed by the detected value y(t) at each time.

The sensor 12 is a membrane-type surface stress (MSS: Membrane-type Surface Stress) sensor. The MSS sensor has, as a receptor, a functional film to which molecules adhere, and a stress generated in a support member of the functional film changes due to attachments and detachments of odor molecules to the functional film. The MSS sensor outputs a detected value based on this change in this stress. The sensor 12 is not limited to the MSS sensor, and may be any one that outputs the detected value based on a variation in a physical quantity related to a viscoelasticity and a dynamic property (a mass, a moment of inertia, or the like) of a member of the sensor 12 that occurs in response to attachments and detachments of the molecules with respect to the receptor. For instance, one of various types of sensors may be employed, such as a cantilever type, a membrane type, an optical type, a piezo, a vibration response, and the like.

For the sake of explanation, sensing by the sensor 12 is modeled as follows.
(1) The sensor 12 is exposed to a target gas containing k types of molecules.
(2) A concentration for each of the k types of molecules in the target gas is a constant ρₖ.
(3) A total of n molecules can be adhered to the sensor 12.
(4) The number of the molecules k attached to the sensor 12 at a time t is denoted by nₖ(t).

In this case, a change in the number nₖ(t) of the molecules k attached to the sensor 12 over time can be formulated as follows.

$$\begin{matrix}[ Math 1 ] & \\ \frac{{dn}_{k} ( t )}{dt} = {α}_{k} {ρ}_{k} - {β}_{k} {n}_{k} ( t ) & ( 1 )\end{matrix}$$

Each of a first term and a second term on a right side of the above formula (1) represents an increase amount of the molecules k per unit time (a number of the molecules k newly attaching to the sensor 12) and a decrease amount of the molecules k per unit time (a number of the molecules k detaching from the sensor 12). Moreover, αₖ denotes a rate constant representing a rate at which the molecules k attach to the sensor 12, βₖ denotes a rate constant representing a rate at which the molecules k detach from the sensor 12.

Here, since the concentration ρₖ is constant, the number nₖ(t) of the molecules k at the time t from the above formula (1) can be formulated as follows.

$$\begin{matrix}[ Math 2 ] & \\ {n}_{k} ( t ) = {n}_{k}^{*} + ( {n}_{k} ( {t}_{0} ) - {n}_{k}^{*} ) {e}^{- {β}_{k} t} & ( 2 )\end{matrix}$$

$$however , {n}_{k}^{*} = \frac{{β}_{k} {ρ}_{k}}{{α}_{k}}$$

Furthermore, assuming that no molecule is attached to the sensor 12 at a time to (an initial state), nₖ(t) is expressed as follows.

[Math 3]

nₖ(t)=n*ₖ(1−e^{-β}^{ₖ}^{t}) (3)

The detected value of the sensor 12 is determined by the stress exerted on the sensor 12 by the molecules contained in the target gas. Accordingly, it is considered that a stress exerted on the sensor 12 by a plurality of molecules can be represented by a linear sum of stresses generated by individual molecules. However, it is considered that a stress generated by each molecule varies depending on a type of the molecule. That is, a contribution of the molecule with respect to the detected value of the sensor 12 differs depending on the type of the molecule.

Therefore, the detected value y(t) of the sensor 12 can be formulated as follows.

$$\begin{matrix}[ Math 4 ] & \\ \begin{matrix}y ( t ) = {\sum }_{k = 1}^{K} {γ}_{k} {n}_{k} ( t ) \\ = \{ \begin{matrix}{ξ}_{0} - {\sum }_{k = 1}^{K} {ξ}_{k} {e}^{- {β}_{k} t} & ( rising case ) \\ {\sum }_{k = 1}^{K} {ξ}_{k} {e}^{- {β}_{k} t} & ( falling case )\end{matrix}\end{matrix} & ( 4 )\end{matrix}$$

$$however , {ξ}_{k} = \frac{{γ}_{k} {α}_{k} {ρ}_{k}}{{β}_{k}} ( k = 1 , … , K ) , {ξ}_{0} = {\sum }_{k = 1}^{K} {ξ}_{k}$$

Here, both γₖ and ξₖ represent contributions of a molecule k with respect to the detected value of the sensor 12. Note that the “rising case” refers to a case of exposing the sensor 12 to the target gas, and the “falling case” refers to a case of removing the target gas from the sensor 12. Note that an operation of removing the target gas from the sensor is performed, for instance, by exposing the sensor to a gas called purge gas.

Here, in a case where the time series data Y obtained by the sensor 12 in which the target gas is sensed can be decomposed as in the above formula (4), it is possible to grasp the types of the molecules contained in the target gas and a ratio of each of various types of the molecules contained in the target gas. That is, by the decomposition represented by the formula (4), data representing features of the target gas, that is, a feature amount of the target gas can be obtained.

Therefore, the odor measurement apparatus 10 acquires the time series data Y output by the sensor 12, and decomposes as expressed in the following formula (5).

$$\begin{matrix}[ Math 5 ] & \\ y ( t ) = {\sum }_{i = 1}^{m} {ξ}_{i} f ( {θ}_{i} ) & ( 5 )\end{matrix}$$

Here, θᵢ denotes a time constant or a rate constant with respect to a magnitude of a change in an amount of the molecules adhering to the sensor 12 over time. ξₖ denotes a contribution value representing a contribution of the feature constant θᵢ to the detected value of the sensor 12.

As a feature constant θ, it is possible to adopt the aforementioned rate constant β and a time constant τ which is an inverse of the rate constant. For each case where β and τ are used as the feature constant θ, the formula (5) can be expressed as follows.

$$\begin{matrix}[ Math 6 ] & \\ y ( t ) = {\sum }_{i = 1}^{m} {ξ}_{i} {e}^{- {β}_{i} t} & ( 6 )\end{matrix}$$

$$y ( t ) = {\sum }_{i = 1}^{m} {ξ}_{i} {e}^{- t / {τ}_{i}} \qquad ( 7 )$$

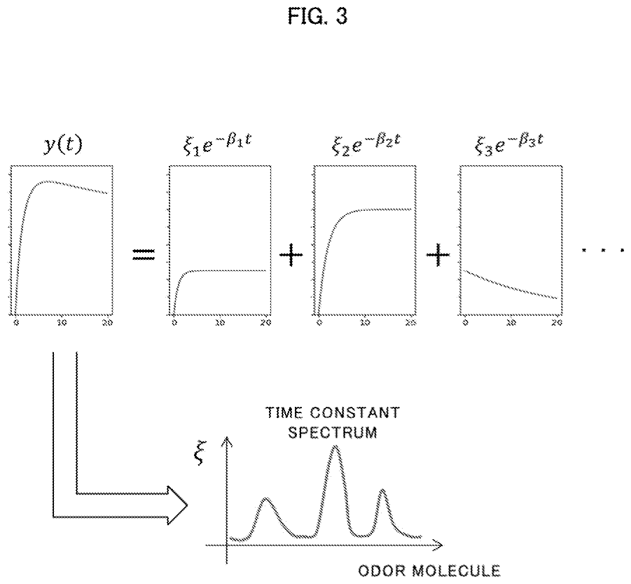

Hereinafter, for convenience of explanation, it is assumed that the time series data Y are represented by the formula (6). As illustrated in FIG. 3, the time series data Y(t) can be expressed as a linear sum of components of each molecule. Therefore, the target gas, that is, an odor of an object can be represented by a graph (hereinafter, referred to as a “time constant spectrum”) taking odor molecules on a horizontal axis and taking a contribution value of each molecule on a vertical axis as illustrated in FIG. 3. In the time constant spectrum, the horizontal axis indicates a dimension of the odor molecules contained in the target gas, and the vertical axis indicates a rate of the odor molecules for each type contained in the target gas, that is, the rate of the odor molecules for each of types which form an odor of the target gas. Accordingly, by analyzing the time constant spectrum, it is possible to investigate what kinds of components the odor of the object is composed of. The odor measurement apparatus 10 outputs the time constant spectrum as odor data for each object. Although the following describes a case of using the time constant spectrum as original data of the odor data, it may be used, raw waveform data before the above-described time constant spectrum is generated may be used as the original data.

[Data Augmentation Apparatus]

(Basic Principle)

As described above, since the time constant spectrum (hereinafter, also referred to as a “TS”) indicates a rate of each odor molecule in a target gas, a model for predicting an object based on features of odor data can be created by machine learning, or the like. Here, since the TS varies depending on an environment such as temperature or humidity, in order to be able to predict in various environments, it is necessary to measure odor data for each environment with different temperature or humidity and to prepare training data for training a model. However, a huge amount of time and a considerable effort are required to prepare training data for all environments by measurement. Therefore, a large number of the training data are prepared by performing a data augmentation for the odor data obtained by the measurement in a specific environment, and by artificially creating sets of odor data in environments with different temperature or humidity.

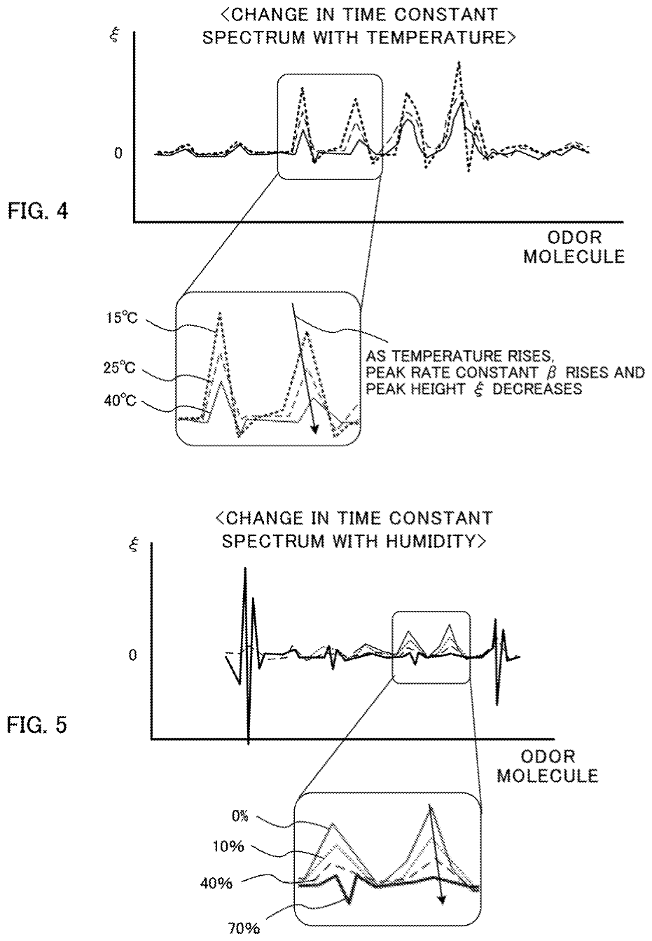

From changes in waveforms of the TS (hereinafter also referred to as “TS waveforms”) respectively obtained in the different environments, it is possible to qualitatively know an effect due to the change in the temperature or the humidity on each TS waveform. FIG. 4 illustrates an example of the change in the TS waveform with the temperature. A horizontal axis indicates a dimension of the odor molecule, and a vertical axis indicates a rate of each odor molecule. FIG. 4 illustrates the TS waveform in a case where the temperature is changed to 15° C., 25° C., and 40° C. while a flow rate of a gas (hereinafter, also referred to as “flow rate”) and the humidity applied to the sensor 12 are constant. As the temperature rises, a rate constant β of a peak of the TS waveform rises, and a height ξ of the peak decreases. Accordingly, with increasing temperature, the TS waveform is shifted in a horizontal axis direction and the level is reduced.

FIG. 5 illustrates an example of the change in the TS waveform with the humidity. A horizontal axis indicates the dimension of the odor molecule, and a vertical axis indicates a rate ξ for each odor molecule. In an example in FIG. 5, the TS waveform is depicted in a case where the humidity is changed to 0%, 10%, 40%, and 70% while the temperature and the flow rate of the gas are constant. Similar to the case of the temperature, the TS waveform shifts in a horizontal axis direction and the level decreases, with increasing humidity.

Therefore, a linear transformation which gives the change of the waveform as mentioned above is obtained, and augmented data are generated based on the original data of odor data by using this linear transformation. In detail, the data augmentation apparatus 20 performs the linear transformation that shifts the TS waveform of the input original data in the horizontal axis direction, and changes a level in response to a change in the temperature or the humidity, so as to generate the augmented data.

(Hardware Configuration)

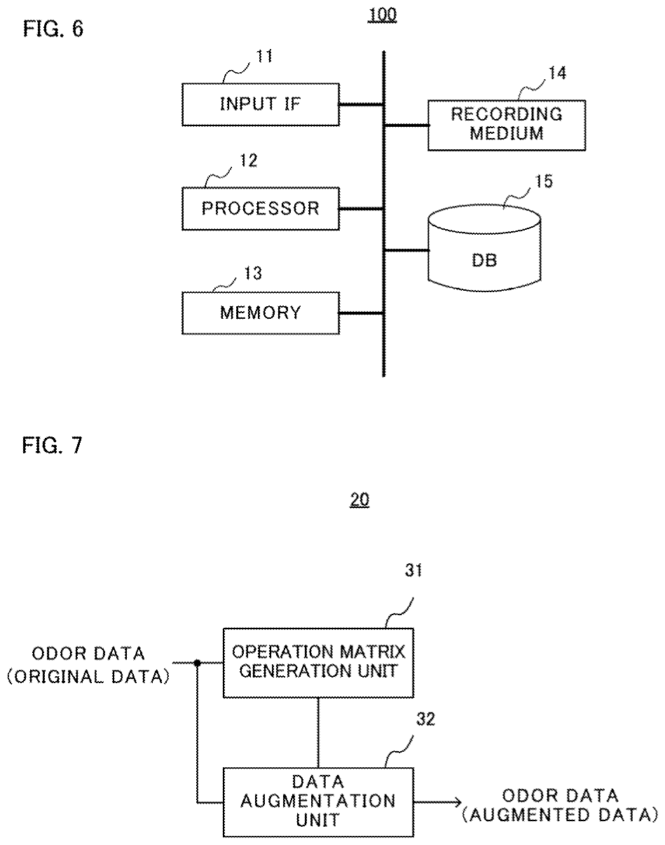

FIG. 6 is a block diagram illustrating a hardware configuration of the data augmentation apparatus 20. As illustrated in FIG. 6, the data augmentation apparatus 20 includes an input IF (InterFace) 21, a processor 22, a memory 23, a recording medium 24, and a database (DB) 25.

The input IF 21 inputs and outputs odor data. In detail, the input IF 21 is used to acquire original data of the odor data from the DB 5 and to store, in the DB 5, augmented data generated by the data augmentation apparatus 20. The processor 22 is a computer such as a CPU (Central Processing Unit) and controls the entire data augmentation apparatus 20 by executing programs prepared in advance. Specifically, the processor 22 executes a data augmentation process, which will be described later.

The memory 23 is formed by a ROM (Read Only Memory), a RAM (Random Access Memory), and the like. The memory 23 stores various programs to be executed by the processor 22. The memory 23 is also used as a working memory during executions of various processes by the processor 22.

The recording medium 24 is a non-volatile and non-transitory recording medium such as a disk-shaped recording medium, a semiconductor memory, or the like, and is formed to be detachable from the data augmentation apparatus 20. The recording medium 24 records various programs executed by the processor 22. When the data augmentation apparatus 20 executes various types of processes, programs recorded on the recording medium 24 are loaded into the memory 23 and executed by the processor 22.

The DB 25 stores data input from an external apparatus including an input IF 21. Specifically, the DB 25 temporarily stores the odor data acquired from the DB 5.

(Functional Configuration)

FIG. 7 is a block diagram illustrating a functional configuration of the data augmentation apparatus. The data augmentation apparatus 20 includes an operation matrix generation unit 31 and a data augmentation unit 32. The operation matrix generation unit 31 generates an operation matrix O for generating augmented data based on the original data of the odor data. The data augmentation unit 32 generates the augmented data using the original data of the odor data and the operation matrix O.

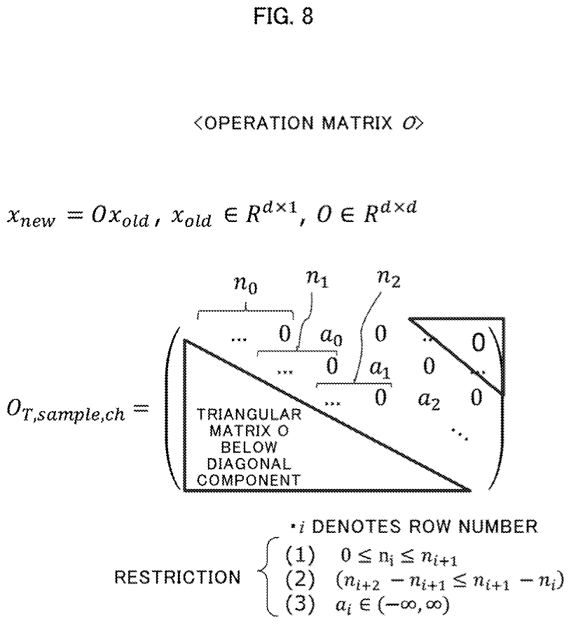

FIG. 8 illustrates an example of the operation matrix O. The operation matrix O performs the linear transformation on the original data measured at a particular temperature or humidity to produce augmented data which are odor data at a different temperature or humidity. For convenience, the following description is an example of generating the augmented data at the different temperatures.

Now, assuming that the original data of the odor data are represented by x_{old}, the operation matrix is denoted by O, and the augmented data are represented by x_{new}, the augmented data can be obtained by the following equation.

X_{new}=Ox_{old}

Here, the original data x_{old} and the augmented data x_{new} are represented by a d×1 dimensional vector (matrix), and the operation matrix O represented by a d×d dimensional vector (matrix).

As illustrated in FIG. 8, the operation matrix O represents that all elements in a triangular matrix below a diagonal component indicate “0”. In elements of each row above the diagonal component of the operation matrix O, first nᵢ columns indicate “0”, a following column indicates “aᵢ”, and subsequent columns indicates “0”. Here, “nᵢ” indicates a shift amount in the horizontal axis direction of the TS waveform by the linear transformation, and “aᵢ” indicates the level change rate of the TS waveform. By setting an appropriate value for the shift amount “nᵢ” and the level change rate “aᵢ”, the TS waveform is shifted in the horizontal axis direction by the operation matrix O, and the linear transformation is performed to change the level.

Note that restrictions (1) through (3) illustrated in FIG. 8 are given to the operation matrix O. The restriction (1) indicates that the lower position the row in the operation matrix O, the greater the shift amount. The restriction (2) indicates that the smaller the rate constant β, the larger the shift amount. The restriction (3) indicates that the level change rate aᵢ is within a range of “−∞ to ∞”. Note that the restriction (2) is not mandatory and is optional.

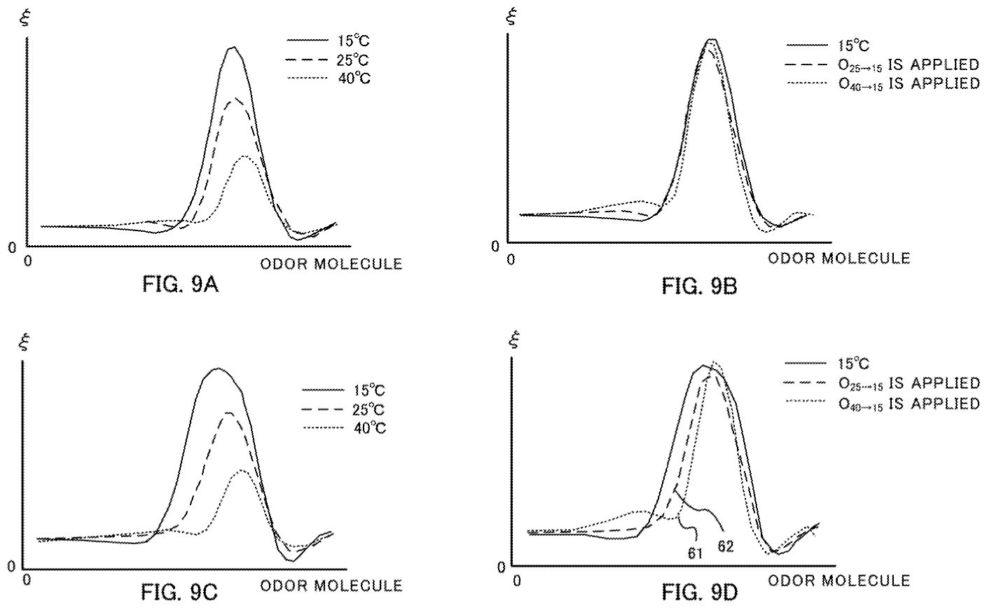

FIG. 9A through FIG. 9D illustrate an example of a data augmentation using the operation matrix O. FIG. 9A illustrates TS waveforms in a case where the original data are measured by the odor measurement apparatus 10 at respective temperature set to 15° C., 25° C., and 40° C. The humidity and the flow rate are constant. Using the TS waveforms in FIG. 9A, as illustrated in FIG. 9B, an operation matrix O_{40→15} is determined so that a position and a magnitude of a peak of the TS waveform at 40° C. coincide with a position and a magnitude of a peak of the TS waveform at 15° C. That is, the operation matrix O_{40→15} is determined in that the TS waveform of 40° C. is regarded as source data and the TS waveform of 15° C. is regarded as the target data. In this case, a shift amount n_{40→15}=2 is acquired, and a level change rate a_{40→15}=2.5 is acquired. In the same manner, an operation matrix O_{25→15} is determined so that a position and a magnitude of a peak of the TS waveform at 25° C. coincide with the position and the magnitude of the peak of the TS waveform at 15° C. That is, the operation matrix O_{25→15} is determined in that the TS waveform of 25° C. is regarded as the source data and the TS waveform of 15° C. is regarded as the target data. In this case, a shift amount n_{25→15}=1 is acquired, a level change rate a_{25→15}=1.3 is acquired.

Next, the operation matrices O_{40→15} and O_{25→15} thus obtained are applied to another set of original data illustrated in FIG. 9C in order to generate augmented data. FIG. 9C illustrates respective TS waveforms in a case of 15° C., 25° C., and 40° C. in the temperature. The humidity and the flow rate are constant. FIG. 9D illustrates respective waveforms of sets of obtained augmented data. In detail, the TS waveform of 15° C. in FIG. 9D corresponds to the TS waveform of 15° C. in FIG. 9C. A waveform 61 in FIG. 9D is obtained by multiplying the operation matrix O_{40→15} to the TS waveform of 40° C. in FIG. 9C. Moreover, the waveform 62 of FIG. 9D is obtained by multiplying the operation matrix O_{25→15} to the TS waveform of 25° C. in FIG. 9C. As illustrated in FIG. 9D, a position and a magnitude of a peak of each set of data of 15° C., 25° C., and 40° C. are substantially matched. Accordingly, it can be seen that the linear transformation using the operation matrix O, it is possible to generate augmented data with a different temperature based on the original data.

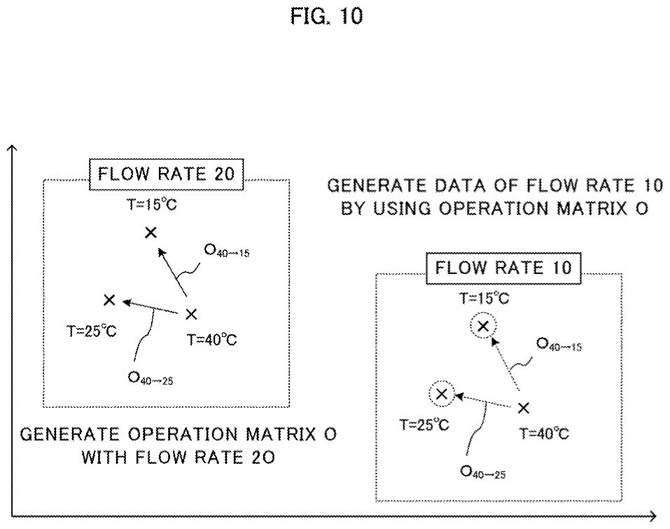

FIG. 10 schematically illustrates an example of the data augmentation. First, the operation matrices O_{40→15} and O_{40→25} are generated using the TS waveforms at 15° C., 25° C. and 40° C. obtained in an environment with a flow rate of 20 sccm. Next, the TS waveform with the temperature of 40° C. is measured in an environment with a flow rate of 10 sccm, and TS waveforms are generated with respective temperatures of 15° C. and 25° C. by applying the above operation matrix O_{40→15} and O_{25→15} to the measured TS waveform. By this data augmentation, without actually performing measurement, it is possible to generate the TS waveforms corresponding to the respective temperatures 15° C. and 25° C. with the flow rate 10 sccm, by a calculation using the operation matrix O.

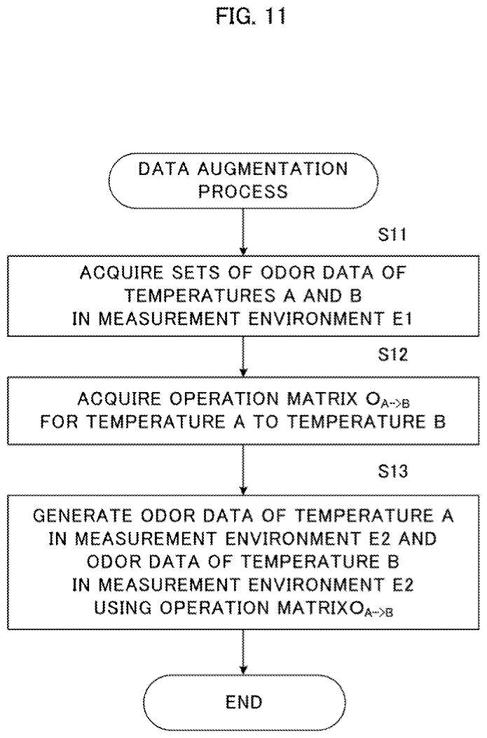

FIG. 11 is a flowchart illustrating a data augmentation process. This process is realized by the processor 22 illustrated in FIG. 6, which executes a program prepared in advance. First, the operation matrix generation unit 31 acquires sets of odor data, which are measured at a plurality of temperatures A and B in a specific measurement environment E1 (step S11). Next, the operation matrix generation unit 31 generates an operation matrix O_{A→B} in order to generate augmented data from the sets of the odor data corresponding to the temperatures A and B (step S12). Subsequently, the data augmentation unit 32 generates odor data (augmented data) of the temperature B in a measurement environment E2 by using odor data (original data) which are measured at the temperature A in the measurement environment E2 different from the specific measurement environment E1 (step S13). After that, the data augmentation process is terminated.

Next, a method for generating the operation matrix O will be described in detail.

(A) First Method

In a first method, all shift amounts nᵢ of the operation matrix O are the same value and all level change rates aᵢ are the same values. In a case where the source data used for generating the operation matrix O are denoted by x_{source}, and the target data are denoted by x_{target}, the operation matrix O is generated so that a product Ox_{source} of the source data x_{source} and the operation matrix O is closer to the target data x_{target}.

Now, a difference d is defined as follows and O(n, a) is acquired so as to minimize the difference d.

d=∥x_{target}−Ox_{source}∥

where ∥⋅∥ represents a norm.

In detail, first, an initial value dₘᵢₙ of the difference d is set, the level change rate a and the difference d are calculated by the following formulas.

a=argmin∥x_{target}−O(n a)x_{source}∥

d=∥x_{target}−O(n, a)x_{source}∥
Then, −a=a and dₘᵢₙ=d when dₘᵢₙ>d.
By repeating this process a predetermined number of times, a combination of n and a is acquired so that the difference d is minimized.

In the formula of the level change rate a, in order for a value of the level change rate a not to be excessive, a regularization term may be added as follows.

a=argmin∥x_{target}−O(n a)x_{source}∥+λ∥a∥,

where “λ” is an arbitrary coefficient.

(B) Second Method

In a second method, each shift amount nᵢ of the operation matrix O is a different value and each level change rate aᵢ is a different value. In a case where the source data used for generating the operation matrix O are denoted by x_{source} and the target data are denoted by x_{target}, the operation matrix O is generated so that the product Ox_{source} of the source data x_{source} and the operation matrix O is closer to the target data x target.

Similar to the first method, the difference d is defined as follows.

d=∥x_{target}−Ox_{source}∥

where ∥⋅∥ represents a norm. Then, O(n, a) is obtained so as to approach the difference d to “0”, and n is obtained so as to minimize a parameter Σᵢ|aᵢ|. In the second method, both the shift amount n and the level change rate a are vectors (may be different vectors depending on i).

In the second method, the solution is not uniquely determined even in a case where the norm becomes “0” due to x_{target} dimensions of the level change rate a. Accordingly, by enumerating the shift amount n, n is acquired so that the parameter Σᵢ|aᵢ| is minimized. At this time, for the shift amount n, it is sufficient to determine a realistic range based on an actual TS waveform and perform a search within the range.

(Modification)

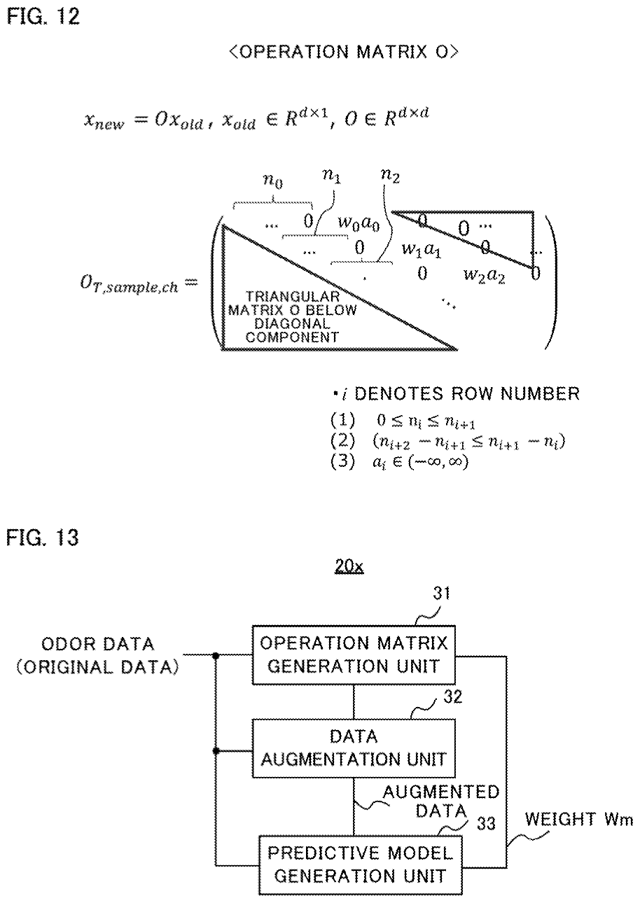

Next, a modification of the first example embodiment will be described. In the modification, a weight is added to the level change rate a of the operation matrix O. FIG. 12 is a diagram for explaining the operation matrix O according to the modification. As illustrated, the level change rate aᵢ is multiplied with a weight wᵢ. In the operation matrix O, by changing the level change rate a, it is possible for the product Ox_{source} of the source data and the operation matrix to be closer the target data x_{target}; however, it is not always necessary that the product of the source data and the operation matrix exactly matches the waveform of the target data. Accordingly, it is determined in advance which portion of the waveform of the target data is to be exactly matched and which portion of the waveform of the target data may be slightly deviated. Next, the weight w is adjusted so that a degree of matching with respect to the portion to be accurately matched (hereinafter, also referred to as “target portions”) is increased among the portions of the waveform of the target data. For instance, in a case where a portion of each peak of the target data is important and is regarded as the target portion, the weight w is determined so that the product Ox_{source} of the source data and the operation matrix exactly matches with the target data x_{target} at each peak portion of the TS waveform. By this determination, it is possible to generate augmented data, which precisely represents each target portion in the TS waveforms.

FIG. 13 is a block diagram illustrating a functional configuration of a data augmentation apparatus 20x according to the modification. The data augmentation apparatus 20x includes an operation matrix generation unit 31, a data augmentation unit 32, and a predictive model generation unit 33. The operation matrix generation unit 31 generates an operation matrix O from sets of odor data which are respectively measured at a plurality of temperatures in a specific measurement environment. Note that the operation matrix O is intended to use the weight w as illustrated in FIG. 12. The data augmentation unit 32 uses an original data measured in another measurement environment and the operation matrix O, and generates augmented data at another temperature in that measurement environment.

The predictive model generation unit 33 generates a predictive model for predicting an object or the like from odor data using machine learning or the like. In detail, the predictive model generation unit 33 trains the predictive model using the original data and the augmented data generated by the data augmentation unit 32. At this time, the predictive model generation unit 33 generates each weight Wm indicating an important portion in the prediction based on the odor data, that is, the target portion of the TS waveform. For instance, in a case where the predictive model is the linear model, each coefficient of the predictive model can be used as the weight Wm. The weight Wm is input to the operation matrix generation unit 31.

The operation matrix generation unit 31 normalizes the weight Wm input from the predictive model generation unit 33 and sets the normalized weight Wm to a weight w of the operation matrix O illustrated in FIG. 12. Next, the operation matrix generation unit 31 generates augmented data using the set weight w, and outputs the augmented data to the predictive model generation unit 33. The predictive model generation unit 33 performs learning using the newly input augmented data, and updates the weight Wm of the predictive model. Accordingly, the data augmentation apparatus 20x repeats the above-described process until a predetermined convergence condition is provided, and employs the weight w of the operation matrix O at a time when the convergence condition is provided.

According to the above modification, it is possible to inherit features of the target portion which is important in the prediction using the odor data to the augmented data.

### Second Example Embodiment

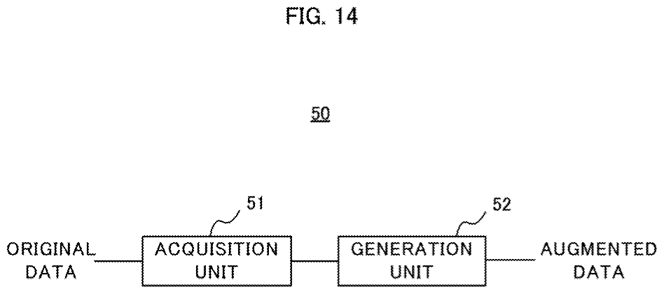

FIG. 14 is a block diagram illustrating a functional configuration of a data generation apparatus according to a second example embodiment. The data generation apparatus 50 of the second example embodiment includes an acquisition unit 51, and a generation unit 52. The acquisition unit 51 acquires original data which are odor data measured in a specific environment. The generation unit 52 performs the linear transformation on the original data, and generates augmented data which are odor data in an environment where a temperature or humidity is different from those in the environment described above.

A part or all of the example embodiments described above may also be described as the following supplementary notes, but not limited thereto.

(Supplementary Note 1)

1. A data generation apparatus comprising:

an acquisition unit configured to acquire original data which are odor data measured in a specific environment; and

a generation unit configured to perform a linear transformation with respect to the original data, and generate augmented data which are odor data in an environment where temperature and humidity are different from that in the specific environment.

(Supplementary Note 2)

2. The data generation apparatus according to supplementary note 1, wherein

each set of the odor data represents features of an object with a waveform that indicates a rate of each of a plurality of odor molecules,

the waveform indicates the plurality of odor molecules on a horizontal axis and the rate of each of the plurality of odor molecules on a vertical axis, and

the generation unit generates the augmented data by performing the linear transformation with respect to a waveform of the original data.

(Supplementary Note 3)

3. The data generation apparatus according to supplementary note 2, wherein the linear transformation shifts the waveform of the original data in a horizontal axis direction and changes a level.

(Supplementary Note 4)

4. The data generation apparatus according to supplementary note 3, wherein the generation unit generates a vector representing the augmented data by multiplying a vector representing the waveform of the original data with an operation matrix expressing the linear transformation.

(Supplementary Note 5)

5. The data generation apparatus according to supplementary note 4, wherein the operation matrix shifts each of elements of the vector representing the waveform of the original data and changes the level with the same level change rate.

(Supplementary Note 6)

6. The data generation apparatus according to supplementary note 4, wherein the operation matrix shifts each of elements of the vector representing the waveform of the original data with the same shift amount or a different shift amount, and changes the level with the same level change rate or a different level change rate.

(Supplementary Note 7)

7. The data generation apparatus according to supplementary note 4, wherein the operation matrix shifts each of elements of the vector representing the original data with the same shift amount or a different shift amount, and changes the level with a level change rate which is weighted with the same weight or a different weight.

(Supplementary Note 8)

8. The data generation apparatus according to supplementary note 7, further comprising

a predictive model generation unit configured to generate a predictive model that predicts an object based on odor data by using the original data and the augmented data; and

a weight determination unit configured to determine a weight for weighting the level change rate based on the weight of the predictive model.

(Supplementary Note 9)

9. A data generation method, comprising:

acquiring original data which are odor data measured in a specific environment; and

performing a linear transformation with respect to the original data, and generating augmented data which are odor data in an environment where temperature and humidity are different from that in the specific environment.

(Supplementary Note 10)

10. A recording medium storing a program, the program causing a computer to perform a process comprising:

acquiring original data which are odor data measured in a specific environment; and

performing a linear transformation with respect to the original data, and generating augmented data which are odor data in an environment where temperature and humidity are different from that in the specific environment.

While the disclosure has been described with reference to the example embodiments and examples, the disclosure is not limited to the above example embodiments and examples. Various modifications that can be understood by those skilled in the art can be made to the structure and details of the present disclosure within the scope of the present disclosure.

### DESCRIPTION OF SYMBOLS

5, 6 Database (DB)

10 Odor measurement apparatus

12 Sensor

20, 20x Data augmentation apparatus

22 Processor

23 Memory

31 Operation matrix generation unit

32 Data augmentation unit

33 Predictive model generation unit

What is claimed is:

1. A data generation apparatus comprising:
a memory storing instructions; and
one or more processors configured to execute the instructions to:
acquire original data which are odor data measured in a specific environment; and
generate augmented data by performing a linear transformation with respect to the original data, the augmented data being odor data in an environment where temperature and humidity are different from that in the specific environment.

2. The data generation apparatus according to claim 1, wherein
each set of the odor data represents features of an object with a waveform that indicates a rate of each of a plurality of odor molecules,
the waveform indicates the plurality of odor molecules on a horizontal axis and the rate of each of the plurality of odor molecules on a vertical axis, and
the processor generates the augmented data by performing the linear transformation with respect to a waveform of the original data.

3. The data generation apparatus according to claim 2, wherein the linear transformation shifts the waveform of the original data in a horizontal axis direction and changes a level.

4. The data generation apparatus according to claim 3, wherein the processor generates a vector representing the augmented data by multiplying a vector representing the waveform of the original data with an operation matrix expressing the linear transformation.

5. The data generation apparatus according to claim 4, wherein the operation matrix shifts each of elements of the vector representing the waveform of the original data and changes the level with the same level change rate.

6. The data generation apparatus according to claim 4, wherein the operation matrix shifts each of elements of the vector representing the waveform of the original data with the same shift amount or a different shift amount, and changes the level with the same level change rate or a different level change rate.

7. The data generation apparatus according to claim 4, wherein the operation matrix shifts each of elements of the vector representing the original data with the same shift amount or a different shift amount, and changes the level with a level change rate which is weighted with the same weight or a different weight.

8. The data generation apparatus according to claim 7, wherein the processor is further configured to
generate a predictive model that predicts an object based on odor data by using the original data and the augmented data; and
determine a weight for weighting the level change rate based on the weight of the predictive model.

9. A data generation method, comprising:
acquiring original data which are odor data measured in a specific environment; and
generating augmented data by performing a linear transformation with respect to the original data, the augmented data being odor data in an environment where temperature and humidity are different from that in the specific environment.

10. A non-transitory computer-readable acquiring original data which are odor data measured in a specific environment; and
generating augmented data by performing a linear transformation with respect to the original data, the augmented data being odor data in an environment where temperature and humidity are different from that in the specific environment.
